(19) United States
(12) Patent Application Publication
(10) Pub. No.: US 2014/0236107 A1
(43) Pub. Date: Aug. 21, 2014

(54) ABSORBENT HYGIENIC OR PERSONAL CARE PRODUCT HAVING A STRIP-LIKE COMPONENT WITH A CONTENT OF AT LEAST ONE HEAVY METAL PRESENT IN ELEMENTAL OR IONIC FORM

(71) Applicant: Birgit Riesinger, Munster (DE)

(72) Inventor: Birgit Riesinger, Munster (DE)

(21) Appl. No.: 14/178,773

(22) Filed: Feb. 12, 2014

Related U.S. Application Data
(63) Continuation of application No. PCT/EP2012/066349, filed on Aug. 22, 2012.

(30) Foreign Application Priority Data
Aug. 23, 2011 (DE) 10 2011 052 941.1
Dec. 14, 2011 (DE) 10 2011 056 429.2
Feb. 22, 2012 (DE) 10 2012 101 413.2
Feb. 27, 2012 (DE) 10 2012 101 559.7

(51) Int. Cl. A61L 15/18; A61F 13/84

(52) U.S. Cl. CPC A61L 15/18; A61F 13/8405
USPC 604/319; 604/360

(57) ABSTRACT

The present invention relates to an absorbent hygienic or personal care product having an absorption body with a content of super-absorbent polymer based on acrylates, wherein said hygienic or personal care product further comprises a strip-like component having a content of at least one heavy metal present in elemental or ionic form.

### CROSS-REFERENCE TO RELATED APPLICATIONS

The present application claims priority from and is a continuation from PCT Application No. PCT/EP2012/066349, filed Aug. 22, 2012, which claims priority from German Patent Application No. DE 10 2011 052 941.1, filed Aug. 23, 2011, German Patent Application No. DE 10 2011 056 429.2, filed Dec. 14, 2011, German Patent Application No. DE 10 2012 101 413.2, filed Feb. 22, 2012, and German Patent Application No. DE 10 2012 101 559.7, filed Feb. 27, 2012, all of which are incorporated herein by reference in their entireties.

### BACKGROUND

The present invention relates to an absorbent hygienic or personal care product having a content of at least one heavy metal present in elemental or ionic form.

In hygiene and wound care it is becoming more common to deal with aggressive and resistant microorganisms. In particular, in chronic, poorly healing wounds, as often found in diabetics, for example, infections play an important role. This problem has intensified in the recent past by antibiotic-resistant pathogens. In particular, multi-resistant germs such as, for example, vancomycin-resistant enterococci, methicillin-resistant Staphylococcus aureus and multidrug-resistant Pseudomonas aeruginosa play in clinical practice today, an often fatal role.

In the U.S., annually, 90,000 ulcer cruris patients with diabetes have amputated their limbs, as there is no other treatment due to infectious complications caused by multidrug-resistant germs

In the case of incontinence products, especially diapers, on the other hand, there is the risk that, in particular, vancomycin-resistant enterococci, who find ideal living conditions in such a product when it is in use, multiply and infest sore skin of the person wearing said product. This is a real danger particularly because incontinence patients are often bedridden and have bedsores (for example, in the lower-spine area) in which a very difficult to treat infection could form in case of an invasion of such resistant germs. Moreover, there is a risk of the existence of vancomycin-resistant enterococci in the digestive tract in such patients because of their sometimes long medical history.

But also for other hygiene and/or wound care products such as, for example, sanitary pads, tampons, incontinence products, diapers, underpads, colostomy bags, colostomy bag inserts, floor mats, surgical towels, Redon bottles, tissues and/or hyperhidrosis products, there is a risk that antibiotic-resistant pathogenic germs proliferate due to the often favorable growth conditions in the same and pose a risk of infection.

Absorbent hygienic or personal care products are known from the prior art, therefore, that contain heavy metals such as silver. EP1656914 to the applicant of the present invention describes an absorption body for absorbing liquid exudate leaking from wounds, having, inter alia, a layer of liquid absorbent textile section with super-absorbent particles contained therein, and an additional amount of an antimicrobial silver-containing substance incorporated into the material of the textile section or the casing.

Due to the fact that according to EP 1656914, the antimicrobial silver-containing substance is “incorporated” into the material of the textile section or casing, substantial production-related and procedural problems may arise under certain circumstances. For example, the precise metering of the silver-containing substance into the material of the casing or the textile section is not easily accomplished. Also, care must be taken that the silver-containing substance does not leave the material of the casing or the textile section uncontrollably. Furthermore, it must be ensured that the silver-containing substance is provided in a sufficiently storable form.

### SUMMARY OF THE INVENTION

Provided herein are systems, methods and compositions for an absorbent hygienic or personal care product having an absorption body with a content of super-absorbent polymers based on acrylates, wherein said hygienic or personal care product further comprises a strip-like component having a content of at least one heavy metal present in elemental or ionic form.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Therefore, it is the object of the present invention to provide an absorbent hygienic or personal care product that does not have the disadvantages mentioned.

This object is achieved with the features of the independent claims. The dependent claims define preferred embodiments.

Thus, there is provided an absorbent hygienic or personal care product having an absorption body with a content of super-absorbent polymers based on acrylates, wherein said hygienic or personal care product further comprises a strip-like component having a content of at least one heavy metal present in elemental or ionic form.

The feature that at least one heavy metal present in elemental or ionic form in form of a strip-like component is provided, results in substantial production-related and procedural advantages, particularly in relation to the precise metering of the silver-containing substance into the material of the casing or of the textile section which is not easy to accomplish, the controllability of the whereabouts of the silver-containing substance and adequate suitability for storage.

All this can be accomplished, for example, by the fact that the strip-like component is purchased from a supplier who meets the specified conditions with manufacturing technology.

Also, in this manner a very high degree of flexibility in product design can be achieved. Thus, said strip-like component having a content of at least one heavy metal present in elemental or ionic form in the product according to the invention:

a) can be arranged in the interior of a liquid permeable casing,

b) form an integral component of a casing,

c) can be applied to the exterior of a casing, and/or

d) may be present as a component detached from the absorption body.

Said hygienic or personal care product is preferably a product selected from the group consisting of wound care products, sanitary pads, tampons, incontinence products, diapers, underpads, colostomy bags, colostomy bag inserts, floor mats, surgical towels, Redon bottles, tissues and/or hyperhidrosis products. Likewise, it can also be a personal care product, such as an ointment, a paste formulation, a gel, or the like.

Hereinafter, the term “wound care product” shall refer, in particular, to a wound dressing, preferably a planar wound dressing or a wound care cloth which is intended for the human medical, dental medical and/or veterinary medicine sector.

Preferably, said wound care product may be present in the form of a swab, a wound fabric, a wound dressing, a wound compress, a wound pad, an optionally elastic wrap, a bandage, a patch or a stocking.

In particular, the term “wound care product” can understood to be an ensemble of different products that are placed in a given arrangement on the wound to be treated. This ensemble can form a physical unit by combining the different products in a common casing or—optionally without casing—are adhesively bonded to one another. However, the ensemble may be present in the form of a kit, in which the various products are optionally arranged by means of a wrap, an adhesive tape, an adhesive strip or a patch in a given arrangement on the wound to be treated.

Super-absorbent polymers (SAP) based on acrylates are plastics that are able to absorb liquids many times their own weight—up to 1000 times. Chemically, these are, for example, copolymers of acrylic acid (propenoic acid, C₃H₄O₂) and sodium acrylate (sodium salt of acrylic acid, NaC₃H₃O₂), wherein the ratio of the two monomers can vary. In addition, a so-called core cross-linker (CXL) is added to the monomer solution, which connects the resulting long-chain polymer molecules in some places with one another by chemical bridges (“cross-linking them”). These bridges make the polymer insoluble in water. Upon penetration of water or aqueous salt solutions in the polymer particle, the polymer particle swells and tightens this network at the molecular level, so that the water cannot escape without help. In the hygienic or personal care product according to the invention the superabsorbent polymers may be present in the form of granules, a powder, a bulk, a pellet, a foam, in the form of fibers, a fiber woven fabric, mat or fleece and/or a fiber wadding.

Alternatively, the super-absorbers can be based on methacrylic acids, acrylamide propanesulfonic acid copolymers, starch-acrylic acid graft polymers, vinyl acetate-acrylic acid ester copolymers, acrylonitrile or acrylamide copolymers.

Absorbent hygienic or personal care products of the kind mentioned are not known from the prior art. Although the use of silver in wound dressings in combination with foams, alginates, charcoal, carboxymethyl cellulose or hydrocolloids is known, the use of super-absorbent polymers based on acrylates is not. The following table 1 lists the known uses from the prior art:

TABLE 1

| Additional component | Product name | Manufacturer |
|---|---|---|
| Foam | Mepilex AG | Mölnlycke |
| | Allevyn AG | Smith & Nephew |
| | Biatain Ag | Coloplast |
| | UrgoCell Silver | Urgo |
| Alginate dressings | Algisite Ag | Smith & Nephew |
| | Suprasorb AG | Lohmann & Rauscher |
| | Askina Calgitrol Ag | BRAUN |
| | SeaSorb Ag | Coloplast |
| | Acticoat Absorbent | Smith & Nephew |
| | Melgisorb AG | Mölnlycke |
| | SILVERCELL | Systagenix |
| | Hydroalginate | |
| | Urgosorb Silver | Urgo (with CMC) |
| Charcoal | ACTISORB Silver | Systagenix |
| | Vliwaktiv AG | Lohmann & Rauscher |
| Carboxymethyl cellulose | AQUACEL Ag | Convatec |
| (CMC) | | |
| | Urgosorb Silver | Urgo (with alginate) |
| Hydrocolloid | Contreet | Coloplast |

Furthermore, the use of a strip-like component having silver is known from the product Kalypto NPD 1000. The latter is a wound dressing for reduced pressure wound treatment, comprising a pad with an underlying silver-containing strip, which consists of a fiber material, which is referred to as super-absorbent. This material is a glycosidic polymer containing a sodium glucosamine—thus, not a super-absorbent polymer based on acrylates.

As the applicant has demonstrated in earlier patent applications, super-absorbent polymers, unlike foams, alginates, charcoal, carboxymethyl cellulose, hydrocolloids or glycosidic polymers containing sodium glucosamine, are able to absorb and bind large amounts of exudate. In doing so, they reduce the content of pathological exudate in the wound, thereby promoting wound healing.

Said super-absorbent polymers bind—also in contrast to foams, alginates, charcoal, carboxymethyl cellulose or hydrocolloids—not only liquids, but also bacteria, proteins and other biomolecules.

This effect ideally complements the antimicrobial and antiviral effect of the heavy metal present in elemental or ionic form. This is the case, since studies have shown that the heavy metals and heavy metal ions, respectively—in particular based on silver or copper—develop their antimicrobial and antiviral properties within a very short time. In doing so, however, this can—especially if the microorganisms are bacteria—result in a release of endotoxins and bacterial pathogenicity factors (especially bacterial hemolysin and leukocidin). These in turn can cause inflammations, allergies, shock (especially anaphylactic shock and/or toxic shock syndrome) and fever (Herxheimer reaction). This is particularly true when copper oxide is applied topically, for example, in the form of a hygienic or personal care product fitted in said manner, such as a wound dressing.

Thus, the hygienic or personal care product according to the invention will not only contribute to reduce the number of pathogens in the wound by absorbing the microorganisms, especially bacteria, by means of SAP and/or lysis of the bacteria, but also absorbs actively the lysates formed, particularly the endotoxins contained therein. The latter also contributes to a relief of the immune system, which otherwise would have to handle and dispose of the resulting endotoxins.

Another advantage of the hygienic or personal care product according to the invention is that it can be used not only for the treatment of wounds that are infected with antibiotic-resistant germs, but that it can also be used in cases in which for other reasons antibiotic therapy is not indicated. This applies, for example, to:
pregnant women, who usually may be treated with only a very limited spectrum of antibiotics (especially erythromycin);
people with antibiotic allergy;
people with liver damage or systemic defects, where there is a risk of metabolic intoxication in case of antibiotic therapy.

Another advantage of the hygienic or personal care product according to the invention is that it is particularly suitable for the treatment of burn wounds, which on the one hand are highly exuding (which is why the use of SAP is sensible), on the other hand, not or hardly supplied with blood, making a systemic antibiotic therapy difficult. Burn wounds, however, are particularly susceptible to infection, in particular to the multi-resistant germs frequently encountered in the hospital setting, so that a therapy with copper and copper ions, respectively, is a new, promising option.

Another advantage of the hygienic or personal care product according to the invention lies in a synergistic reduction of inflammation-related odors, as is common in chronic wounds. Owing to the activity of the copper or copper ions, the metabolic activity of the bacteria responsible for the odor that, in particular, releases butyric acid, is reduced. At the same time, the super-absorbent polymers absorb and bind both already odorants already formed as well as exudate and water from the wound. By draining the wound, the growth conditions for the odor-causing bacteria are worsened, which leads to a further reduction of odors.

Particularly advantageous is the fact that a colloid comprising heavy metals in elemental or ionic form can form by the wound liquid absorbed by the SAP.

This advantageous formation of a colloid is surprising since SAPs are known to bind exudate so effectively that actually no liquid should be available to form the colloid. Apparently, however, gelation of the super-absorbent polymers is sufficient in order to allow colloid formation. This implicit and surprising way of colloid formation has the advantage over deliberately added colloids, for example, in the form of ointments, which are applied to the hygienic or personal care product according to the invention, that the production can be much more efficient and cost-effective.

The super-absorbent polymers may be present in the hygienic or personal care product according to the invention in the form of granules, a powder, a bulk, a pellet, a foam, in the form of fibers, a fiber woven fabric, mat or fleece and/or a fiber wadding.

The super-absorbent particles may be present in powder or granular form having a particle size of between 100 μm and about 1000 μm.

EP 1656914 to the applicant of the present application discloses a disposable absorption body for absorbing liquid exudate oozing from wounds, having a liquid-permeable casing consisting of two side walls and at least one layer of a liquid-absorbent textile section with super-absorbent particles present therein which are contained in the casing. The absorption body in this case comprises a quantity of an anti-microbially active silver-containing substance which is incorporated into the material of the textile portion or casing.

EP1656914 does not disclose that the silver-containing substance is present in a strip-like component. The design according to the invention, according to which the hygienic or personal care product has a strip-like component having a content of at least one heavy metal present in elemental or ionic form—preferably silver or copper—has, however, significant advantages in production and application. Thus, the production and sales and distribution is more cost-effective and more flexible because the strip-like component can be integrated in the hygienic or personal care product as a prefabricated supplier product or delivered as a kit together with the absorption body more easily. Furthermore, there is the possibility to use the strip-like component as primary dressing in the context of the hygienic or personal care product according to the invention.

The strip-like design facilitates, in particular, the integration of it into the casing of the hygienic or personal care product according to the invention, which in turn facilitates placement as close to the wound as possible.

Preferably, it is provided that the at least one heavy metal present in elemental or ionic form is selected from the group consisting of copper, zinc and/or silver.

This definition encompasses in general, in addition to the heavy metals present in elemental form, the salts thereof, preferably the chlorides, sulfates, sulfides, sulfites, or nitrates, metal organic compounds or the sulfonamide thereof (such as silver sulfadiazine).

The antimicrobial properties of silver or silver ions are known to the person skilled in the art and need not be discussed here further.

Copper and copper ions, respectively, deactivates or kills most efficiently a wide range of viruses and microorganisms. In this context, copper or the copper ions acts in various ways.

1) Since they are highly redox-active metal ions, they promote the peroxidation of membrane lipids and thereby damage the cell wall of the microorganism. In this capacity, they also result in a shift of the essential metals from their original binding sites and thus interfere with the metabolism of the microorganism.

2) They damage the genetic material of the microorganism by binding the DNA and thus change its conformation.

3) They damage the proteins of the microorganism, in particular, they cause degradation of sulfhydryl groups, as well as oxidation of certain amino acid residues.

4) They interfere with the oxidative phosphorylation and disrupt the osmotic balance.

In addition, copper and copper ions, respectively, impacts the different biological defense mechanisms of the microorganism in such a way that resistance against antimicrobial agents is practically no longer present. The same goes for zinc.

Preferably, it is also provided that the heavy metals present in elemental or ionic form are applied by coating onto a support material. The carrier material can be, for example, a film, granules, a fiber or yarn. Preferred coating methods are, for example:
Chemical vapor deposition (CVD);
Flame coating (C-CVD);
Physical vapor deposition (PVD);
Plasma-enhanced chemical vapor deposition (PECVD);
Spin coating;
Spraying;
Dip coating;
Vacuum evaporation;
Sputtering.

The current methods are known to the person skilled in the art, and he is therefore able to apply the above methods for the coating of the primary dressing according to the invention with heavy metals present in elemental or ionic form without further inventive step.

Such silver-coated materials are known, for example, from the applications U.S. Pat. No. 6,861,570, U.S. Pat. No. 6,087,549, U.S. Pat. No. 7,005,556, U.S. Pat. No. 7,214,847 and U.S. Pat. No. 7,230,153. However, they do not mention at any point the synergistic benefits associated with super-absorbent polymers.

The coated film or fiber and the coated granules or yarn, respectively is then processed further to form said strip-like component. In the case of granules, this can be done for example by extrusion, in the case of a fiber or yarn by spinning and subsequent felting, weaving or knitting.

Furthermore, preferably it is provided that the heavy metals present in elemental or ionic form are incorporated in a carrier material by co-extrusion.

Here, the heavy metals present in elemental or ionic form can be incorporated in the extrusion process, for example, in the form of colloids, in form of salts (preferably as chloride, sulfate or nitrate), or in the form of organometallic compounds.

It is further preferred that the heavy metals present in elemental or ionic form are present in the form of filaments or fibers and—optionally, processed together with a further material forming a strip-like material.

Conceivably, this may involve in particular spinning and subsequent felting, weaving or knitting. Optionally, this can take place in combination with other fibers or yarns, such as, for example, polyester, polyamide, elastic yarns, etc.

Moreover, said at least one heavy metal present in elemental or ionic form can be supplemented by calcium phosphate (for example in the form of nano-particles made of calcium phosphate that are coated with silver, zinc or copper). The combination of one of the three heavy metals with calcium phosphate is up to 1000 times more lethal than conventional silver preparations for many germs. A crucial factor seems to be that bacteria use the carrier calcium for their metabolism. Said calcium phosphate particles 20 to 50 nanometers in size are taken up as food by the microorganism, and thereby decomposed. Then, thousands of small silver particles 1-2 nanometers in size are released and exert their bacteriostatic effect.

Furthermore, it is preferred that the absorption body comprises a non-woven in addition to a content of super-absorbent polymers.

Preferably, the absorption body may have a substantially flat absorption body made of absorbent material, consisting of an absorbent fleece having super-absorbent polymers dispersed therein. These can be present in the form of granules, a powder, a bulk, a pellet, a foam, in the form of fibers, fiber woven fabric, mat or fleece and/or a fiber wadding.

In this case, the absorption body comprises at least one material which is selected from the group consisting of a mat, in particular of an airlaid made of said yarns or fibers of super-absorbent polymers with incorporated super-absorbent polymers, and/or a loose fill of super-absorbent polymers. Preferably, said airlaid mat may comprise a substantially flat material section made of absorbent material, which, for example is composed of an absorbent fleece made of said fibers with super-absorbent polymers dispersed therein.

This absorption body may correspond to the absorbent pad contained in a wound dressing of the applicant of the present invention such as disclosed, for example, in WO 03094813, WO 2007051599 and WO 0152780 and marketed under the trade name “sorbion sachet”. The disclosure of the documents mentioned is attached to the disclosure of this document in full.

In another embodiment, the absorption body may also form a core, which comprises—optionally, flaky—fibers or yarns made of the super-absorbent polymers and super-absorbent polymers in the form of granules, wherein the granules are glued to or welded on or fibers and yarns, respectively, at several heights, and the granules are distributed over more than 50% of the total design height of at least one section of the core, wherein blended areas of granules and fibers are present. Preferably, the content by weight of the super-absorbent polymer can be in the range of 10-25% by weight. Similar constructions are known from conventional incontinence materials and known just like sanitary pads for their cushioning properties. Around said core a casing may be arranged, which is arranged to overlap in areas, and covered by or comprised of, for example, an adhesive.

Particularly preferably, the absorption body comprises a fleece, preferably a nonwoven or airlaid consisting of super-absorbent fibers (“SAF”, preferably polyacrylates) or contains them as a component. The fibers may be mixed with fluff pulp (cellulose) or polyester fibers, for example. Alternatively or additionally, a layer structure may be provided.

In another embodiment, the absorption body may also contain at least one flat layer comprising fibers or yarns made of super-absorbent polymers to which super-absorbent polymers in granular form are glued. In a preferred embodiment this results in a structure of the body having at least two layers, wherein at least one covering layer is underlying a layer comprising super-absorbent polymers. Optionally a second, flanking covering layer may be provided.

In another embodiment, the absorption body may comprise at least one covering layer made of a strip-like material, preferably a fleece material, on which super-absorbent polymers are applied in a pasty or amorphous form, preferably with an auxiliary material. Also this results in a structure of the body having at least two layers, wherein at least one covering layer is underlying a layer comprising super-absorbent polymers. This can result in very high weight percentages of super-absorbent polymers. Optionally, a second, flanking covering layer can be provided.

In both of the abovementioned embodiments there are no blends of fibers and super-absorbent polymers in the plane; rather, only fixed, adjacent sections of both materials. In a preferred embodiment, the several layers optionally provided may also be compressed physically with one another by rolling, pressing, calendering or similar methods.

Particularly preferably it is provided that the absorption body corresponds in its construction, for example, to commercially available products “sorbion sachet”, “TenderWet”, “Zetuvit”, “Drymax”, “Vliwasorb”, and/or “Curea P”.

Furthermore, it is also preferred that said hygienic or personal care product comprises at least in sections a liquid-permeable casing.

The latter surrounds the absorption body, forms a barrier against solid precipitations, facilitates the passage of other oozed-out substances to the material section made of absorbent material arranged within casing. Preferably, the casing is closed at least partially by a seam.

Preferably, the pores or meshes of the casing are 0.05 mm to 1.0 mm, preferably 0.20 mm to 0.50 mm in size. In general it can be provided that the mean pore size is less than the mean size of the particles containing hydro-active polymers. Furthermore, preferably, it can also be provided that the pores or meshes are limited by the thread or fiber sections which in cross-section through the casing are approximately arc-shaped and with their arc vertices pointing outwardly.

Preferably, the casing is formed of woven or nonwoven composite synthetic fibers such as polypropylene or polyethylene fibers, but also cotton, silk or rayon. Preferably, the casing is made of a woven or nonwoven fabric having at least an area-specific weight of 20 g/m².

Preferably, the casing is made of a hydrophobic material, or the casing material is rendered hydrophobic. The hydrophobic properties of the casing prevent it from adhering to the wound surface and contribute to the wound exudate particles reaching the interior of the casing faster.

It can be provided that the casing comprises, at least in sections, an elastic material, such as, for example, fibers made of Lycra or spandex. This also ensures that the material section can increase in its volume upon liquid uptake and is not limited by the casing.

WO 03094813, WO 2007051599 and WO 0152780 to the applicant of the present invention, for example, disclose absorption bodies of the type mentioned.

The material of the casing may be structured in such a way that the casing has a rough inner surface and a smooth outer surface. Preferably, the rough inner surface of the shell is formed by funnel-shaped perforations, each tapering in the direction of the inner surface and end in a free opening edge (“overhang”). This rough inner surface counteracts the displacements of the content of the casing so that a fixation with adhesive dots is dispensable. Accordingly, the smooth outer surface of the casing material can be formed by arched material sections extending between the perforations. As opposed to a casing material that is flat on both sides such a casing material may be referred to as “three-dimensional”, and is known from, for example, DE 102006017194 to the applicant of the present application, to the disclosure of which reference is made in its entirety.

Particularly preferably, it is provided here that said three-dimensional casing material is laminated to the aforementioned polypropylene fleece. Such an embodiment has improved liquid absorption properties.

Likewise, it can be provided, that the hygienic or personal care product on its side facing away from the wound has a liquid-impermeable clothing protection (“back sheet”).

Furthermore, it is preferred that the hygienic or personal care product according to any one of the preceding claims, wherein said strip-like component having a content of at least one heavy metal present in elemental or ionic form,

a) is arranged in the interior of the liquid-permeable casing,

b) forms an integral component of the casing,

c) is applied to the exterior of the casing, and/or

d) is present as a component detached from the absorption body.

In the latter case, the hygienic or personal care product according to the invention represents a kit consisting of an optionally encased absorption body and a strip-like component detached therefrom having a content of at least one heavy metal present in elemental or ionic form.

Both components may be present in one package (optionally sterile package) or in two separate packages that are jointly delivered or intended for joint use (optionally sterile packages).

In the case where the strip-like component is present as a component that is detached from the absorption body, the strip-like component can be used as a primary dressing which is first placed on the wound, to then apply the optionally encased absorption body in a second step.

Particularly preferably, the absorption body in a plan view onto its flat side has a surface area (F1) which is, in its unwetted state, 3% to 75% smaller than the surface area (F2) of the flattened casing, and is freely movable in the casing or fixed, wherein the casing has, at least in sections, pores which on average are smaller than the unwetted super-absorbent particles.

This ensures that the material section can increase in its volume upon liquid uptake and is not limited by the casing.

Particularly preferably, it is provided that the casing in plain view onto its flat side has a circumferential upper level [‘Oberstand’] protruding over the seam, and the absorption body is free of hard, sharp edges and corners.

Particularly preferably, said hygienic or personal care product has a surface size of 5×10, 5×20, 10×20, 10×10, 10×15 or 15×15 cm.

The area density may be in the range between ≧50 and ≦2000 g/m². Preferably are provided area densities of 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, and/or 2000 each +/−25 g/m².

The thickness may be in the range between ≧2 and ≦50 mm. Preferably are provided thicknesses of 2, 4, 6, 8, 10, 12, 14, 16, 18, 20, 22, 24, 26, 28, 30, 32, 34, 36, 38, 40, 42, 44, 46, 48, and/or 50 each +/−1 mm.

The absorption capacity may be in the range between ≧3 and ≦30 ml of 0.9% sodium chloride/m² at a pressure of 0.2 psi. Preferably are provided values of 3, 4, 5, 6, 7, 8, 9, 10, 11, 12, 13, 14, 15, 16, 17, 18, 19, 20, 21, 22, 23, 24, 25, 26, 27, 28, 29, and/or 30 ml 0.9% sodium chloride/m². Alternatively, the absorption capacity can be in the range between ≧2 and ≦50 g of water/g. Preferably are provided thicknesses of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50 g of water/g.

The total content of super-absorbent polymers may be in the range between ≧5 and ≦100% (Gew./Gew.). Preferred are values of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and/or 100% Gew./Gew.

The tensile strength may be in the range between ≧5 and ≦80 N/5 cm. Preferred are values of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and/or 80 N/5 cm.

The expansibility may be in the range between ≧10 and ≦80%. Preferred are values of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, and/or 80%.

When using super-absorbent fibers, in practice the following types were found to be particularly advantageous, as shown in Table 2:

TABLE 2

| Type | 1 | 2 | 3 | 4 | 5 | 6 |
|---|---|---|---|---|---|---|
| Construct 1 | Layer | 40% | Biocomponent | Layer | 25% | 40% |
| | structure: | polyester | fiber made of | structure: | polyester; | polyester |
| | thermally | short cut | SAF and a | thermally | 75% SAF | short cut |
| | bonded | fiber; 60% | thermoplast | bonded | | fiber; 60% |
| | airlaid with | SAF | | airlaid with | | SAF |
| | laminated | | | laminated | | |
| | nonwoven | | | nonwoven | | |
| Construct 2 | Biocomponent | Needle | Carded, | Biocomponent | Needle | Needle |
| | fiber made of | felting | thermally | fiber made of | felting | felting |
| | SAF and a | | bonded | SAF and a | | |
| | thermoplast + | | nonwoven | thermoplast + | | |
| | fluff pilp | | | fluff pilp | | |
| Type of SAF | 101/6/10 | 102/52/10 | 102/52/10 | 101/6/10 | | |
| fiber | | | | | | |
| Weight (g/m²) | 560 | 540 | 1000 | 350 | 150 | 380 |
| Thickness (mm) | 6 | 5.4 | 20 | 3.5 | 2.4 | 3.8 |
| Absorption | 31.2 l of | >20 g of | >l6 g of | l9.5 l of | >25 g of | >17 g of |
| capacity | water/m² | water/g | water/g or | water/m² | 0.9% | water/g or |
| | | | 16000 g of | | sodium | 6400 g/m² |
| | | | water/m² | | chloride/g | |
| Absorption | 16 | | | 16 | | |
| capacity under | | | | | | |
| pressure (ml of | | | | | | |
| 0.9% sodium | | | | | | |
| chloride/m² at | | | | | | |
| 0.2 psi pressure | | | | | | |
| Total content of | 18 | 40 | 50 | 18 | 75 | 60 |
| super-absorbent | | | | | | |
| polymers (% | | | | | | |
| Gew./Gew.) | | | | | | |
| Tensile strength | | | | | 16 ± 13 | 16 ± 13 |
| (N/5 cm) | | | | | | |
| Expansibility (%) | | | | | 60 ± 18 | 60 ± 18 |

Similarly preferred parameter ranges apply, as mentioned above. The liquid retention may be in the range between ≧5 and ≦100 g/g. Preferred are values of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and/or 100 g/g.

In practice, the following types have been found to be particularly advantageous, as shown in Table 3:

TABLE 3

| Type | 1 | 2 | 3 | 4 | 5 | 6 | 7 | 8 |
|---|---|---|---|---|---|---|---|---|
| Weight | 450 | 300 | 150 | 50 | 100 | 120 | 140 | 440 |
| (g/m²) | | | | | | | | |
| Thickness | 1.3 | 1.2 | 0.9 | 0.7 | 0.7 | 0.76 | 1 | 1.2 |
| (mm) | | | | | | | | |
| Liquid | 28 | 33 | 28 | 15 | 25 | 28 | 11.5 | 38 |
| retention | | | | | | | | |
| (g/g) | | | | | | | | |
| Tensile | 25 | 55 | 20 | 20 | 20 | 20 | 15 | 20 |
| strength | | | | | | | | |
| (N/5 cm) | | | | | | | | |
| Absorption | 45 | 20 | 50 | 20 | 40 | 50 | 28 | 55 |
| capacity | | | | | | | | |
| (g/g) | | | | | | | | |

Furthermore, preferably, it is provided that the casing or the absorption body has punchings, slits, incisions and/or recesses.

They are preferably formed and/or arranged in such a manner to facilitate liquid entry into and liquid uptake by the wound care product—which is particularly advantageous because of the high endotoxin load of the exudate caused by the heavy metals.

Particularly preferably, it is also provided that said absorption body, in addition to a layer comprising super-absorbent polymers, has at least one flanking second layer having fewer or no super-absorbent polymers and the area of the latter protrudes over the former. In this way it is ensured that the layer having super-absorbent polymers can gain volume corresponding to the liquid absorption, without the increase in volume being visible to the outside, because the latter is concealed by the second layer.

In a further preferred embodiment hygiene or body care product further comprises on a wound contact layer.

The term “wound contact layer” refers to a grid- or gauze-like construct that is placed frequently as so-called “primary dressing” directly on a wound optionally before a secondary dressing is applied. Wound contact layers are often referred to as “wound gauzes” and are primarily intended to prevent sticking of the entire wound dressing to the wound. To this end, wound contact layers are made of a material or coated with such material which reduces or prevents adhesion to the wound. Alternatively, also the geometry of the wound contact layer can be configured such that adhesion to the wound is reduced or prevented.

Commercial examples of wound contact layers are, for example, the products “Mepitel” (silicone-coated elastic polyamide net), “Physiotulle”, “Urgotül” (net of polyester fibers soaked with hydrocolloid particles and petroleum jelly) and “ADAPTIC” (smooth rayon knitted fabric impregnated with an oil-in-water emulsion).

The aforementioned products are two-dimensional wound contact layers, which substantially take the form of a gauze or net. A three-dimensional wound contact layer is known under the name “sorbion plus” and described, in particular, in EP2004116 to the applicant of the present invention, reference being made to its contents in its entirety.

Such a three-dimensional wound contact layer has a liquid-permeable, conformable material section made of a thermoplastic, having a first smooth surface, a surface facing away from the smooth surface and a second rough surface. The latter is formed by a plurality of three-dimensional perforations, the walls of which project starting from the first, smooth surface and end in an edge protrusion [‘Randüberstand’] with free edge, by means of which they impart a rough touch to the second surface.

The perforations are made by unilaterally inserted punchings. This results in a rough side with overhangs and a more or less smooth side.

A wound contact layer reliably prevents sticking of the hygienic or personal care product with the wound and wound edge maceration. Therefore, in contrast to the foams known from the prior art, said active element no longer must be cut to fit the shape of the wound, which reduces the workload of medical staff considerably.

Furthermore, preferably, at least one hygienic or personal care product has a casing, which in turn has means which are designed and/or selected in such a way that the casing at least partially can be deformed purposefully by a volume increase of the absorption body caused by a liquid absorption. In principle, such a behavior can be obtained with unidirectionally elastic material, i.e., a material which is stretchable in one direction but not in the direction perpendicular thereto.

Furthermore, preferably, it is provided that at least one wound care product comprises a composition containing at least one nutritional, at least one disinfecting or decontaminating and/or at least one protease inhibiting active agent and/or active complex for the external care and/or treatment of wounds of the human or animal body.

Furthermore, according to the invention, there is provided the use of a hygienic or personal care product according to one of the preceding claims in a negative pressure wound care system.

Such systems are disclosed, for example, in specifications DE 202004017052, WO 2006048246 and DE 202004018245 to the applicant of the present invention, the disclosure of which are to be considered as belonging to the present invention.

The first-mentioned discloses a device for the treatment of wounds using reduced pressure, comprising a gas-tight wound-covering element, which, in the state when applied to the patient's body, forms a space remaining between the respective wound and the wound-covering element, and at least one connector which is in contact with the space and through which the air within said space can be evacuated, wherein at least one planar wound dressing absorbing the wound exudate is underlying the wound covering element, wherein the volume of the wound dressing increases in the course of the absorption process, so that the absorbed wound exudates remain within the wound dressing, and thus below the wound covering element, until the removal of the wound dressing from the patient's body, the wound dressing is at least one layer of a textile section enriched with super-absorbents, which is surrounded by a liquid-permeable casing, and the layer in plan view on its flat side has an area that is 3% to 90% smaller than that of the casing, so that the wound dressing close to its entire filling capacity can approach a circular shape in cross-section.

The second-mentioned discloses a multi-component dressing for treating wounds of the human or animal body with the use of reduced pressure, comprising:

a wound covering element to be attached to skin and mucosa surface, at least one connector which is in contact with the wound space, and through which the substances present in the wound space can be evacuated, wherein the latter comprises super-absorbent polymers, wherein the absorbed wound exudates bound to polymers remain in the wound space until removal from the wound space, wherein the polymers by their binding capacity support mutual synergies with the sub-atmospheric pressures.

The latter discloses a drainage device for treating wounds using reduced pressure, comprising a gas-tight wound covering element consisting of film-like material which is adhesively attached at the skin surface around the wound area when applied to the patient's body, and forms a sealed space remaining between the respective wound and the wound covering element, at least one drainage tube which can be inserted into the space through which the substances present in the space can be evacuated, and at least a wound dressing arranged within the space and absorbing the wound exudates, having at least one layer of a textile section enriched with super-absorbents, which is surrounded by a liquid-permeable casing, wherein the absorbed wound exudates remain within the wound dressing and thus below the wound covering element until removal of the wound dressing from the body of the patient, and wherein the wound covering element has a gas-tightly closable treatment opening through which the wound dressing can be inserted into the space and removed from the space.

The hygienic or personal care product according to the invention can also have a shape adapted to anatomical circumstances. To that end, for example, it can be formed in the form of a sleeve; that can be slipped over the one arm or a leg or a joint, or a bandage in the form of a bandage adapted to the heel, elbow joint or the like.

Here, a combination of microfibers proves especially advantageous since microfibers due to their cushioning effect pleasantly cushion the mask of the attached vacuum system. Moreover, microfibers support the drainage process particularly effectively due to their moisture-conducting properties. Particularly advantageous is the use of a spacer woven fabric, mat or fleece, as described above, comprising microfibers.

Conceivable is, for example, a vacuum system for attachment near the joint, for example, for attachment to the elbow or heel, as is marketed by the company KCI, San Antonio, USA, wherein the polyurethane foam used in this product is replaced by the microfiber according to the invention having a cushioning effect. Replacing the known polyurethane foam by the microfiber according to the invention provides a cost-effective alternative to the well-known material, and also offers compared to the structure of the polyurethane foam a more targeted draining of the wound liquids by a directed design of the microfibers.

According to the invention, there is also provided the use of a hygienic or personal care product as described above for controlling exudate, for cushioning a wound and/or destruction of a biofilm.

According to the invention, there is provided also the use of a hygienic or personal care product as described above or of a kit for the treatment of chronic, acute and/or bleeding wounds, burn wounds and/or wounds generated by trauma, as well as the use of a hygienic or personal care product as described above or of a kit for surgical or post-surgical care or for military purposes.

Furthermore, there is provided a method for the treatment of wounds, in particular chronic wounds, comprising the use of a kit as described above or of a hygienic or personal care product.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. An absorbent hygienic or personal care product having an absorption body with a content of super-absorbent polymers based on acrylates, wherein said hygienic or personal care product further comprises a strip-like component having a content of at least one heavy metal present in elemental or ionic form.

2. The hygienic or personal care product according to claim 1, wherein the at least one heavy metal present in elemental or ionic form is selected from the group consisting of copper, zinc and/or silver.

3. The hygienic or personal care product according to claim 2, wherein said heavy metals present in elemental or ionic form are applied onto a support material by coating.

4. The hygienic or personal care product according to claim 3, wherein said heavy metals present in elemental or ionic form are incorporated in a carrier material by co-extrusion.

5. The hygienic or personal care product according to claim 4, wherein said heavy metals present in elemental or ionic form are present in filament or fiber form and are processed with a further material to form a strip-like material.

6. The hygienic or personal care product according to claim 5, the absorption body having a non-woven in addition to a content of super-absorbent polymers.

7. The hygienic or personal care product according to claim 6, wherein said hygienic or personal care product at least in sections has a liquid permeable casing.

8. The hygienic or personal care product according to claim 7, wherein the strip-like component having a content of at least one heavy metal present in elemental or ionic form is selected from the group consisting essentially of:
a) is arranged in the interior of the liquid permeable casing;
b) forms an integral component of the casing;
c) is applied to the exterior of the casing; and
d) is present as a component detached from the absorption body.

9. The hygienic or personal care product according to claim 8, wherein the absorption body in a plan view onto its flat side has a surface area (F1) which is, in its unwetted state, 3% to 75% smaller than the surface area (F2) of the flattened casing.

10. The hygienic or personal care product according to claim 9, wherein the casing has pores at least in sections, which are smaller on average than the unwetted super-absorbent polymers.

11. The hygienic or personal care product according to claim 10, wherein the casing or the absorption body have punchings, slots, incisions and/or recesses.

12. A use of a hygienic or personal care product according to claim 1 in a reduced pressure wound treatment system.

13. A use of a hygienic or personal care product according to claim 1 for the treatment of chronic, acute and/or bleeding wounds, burns and/or wounds generated by trauma.
